(19) United States
(12) Patent Application Publication
(10) Pub. No.: US 2024/0165338 A1
(43) Pub. Date: May 23, 2024

(54) DRUG DELIVERY DEVICE

(71) Applicant: AMGEN INC., Thousand Oaks, CA (US)

(72) Inventors: Jan Mikael JENSEN, Söborg (DK); Lars EILERTSEN, Söborg (DK)

(73) Assignee: AMGEN INC., Thousand Oaks, CA (US)

(21) Appl. No.: 18/550,647

(22) PCT Filed: Mar. 3, 2022
(86) PCT No.: PCT/US2022/018596

§ 371 Date: Sep. 14, 2023

Related U.S. Application Data
(60) Provisional application No. 63/166,080, filed on Mar. 25, 2021.

(51) Int. Cl. A61M 5/315; A61M 5/20; A61M 5/31

(52) U.S. Cl. CPC A61M 5/31515; A61M 5/20; A61M 2005/2086; A61M 2005/3143; A61M 2205/02

(57) ABSTRACT

A drug delivery device may include a housing defining a longitudinal axis and having an opening and a drug storage container including a barrel, a stopper and a delivery member, the stopper movably positioned within the barrel and the delivery member positioned at a distal end of the barrel and having an insertion end configured to extend at least partially through the opening during a delivery state. The device may also include plunger moveable toward the distal end of the drug storage container to engage the stopper and expel a drug from the drug storage container through the delivery member, the plunger including a body portion and a shock absorbing portion. The device may further include a plunger biasing member coupled with the plunger and configured to urge the plunger toward the distal end of the drug storage container.

### CROSS-REFERENCE TO RELATED APPLICATION

Priority is claimed to U.S. Provisional Patent Application No. 63/166,080, filed Mar. 25, 2021, the entire contents of which are hereby incorporated herein by reference.

### FIELD OF DISCLOSURE

The present disclosure relates to drug delivery devices, and, more particularly, devices for injecting a drug into a patient.

### BACKGROUND

A general aversion to exposed needles, as well as health and safety issues, have led to the development of drug delivery devices which conceal a needle or other insertion member prior to use and which automate various aspects of an injection process. Such devices offer a variety of benefits as compared with traditional forms of drug delivery including, for example, delivery via a conventional syringe.

Many injector systems use coil and other spring structures to provide actuation energy for functions such as needle insertion and/or fluid delivery. The use of springs can offer benefits of simplicity and low cost, but it may have certain limitations. For example, there is a linear relationship between force and displacement in spring actuators. To provide sufficient energy for drug delivery at the end of plunger stroke, an excessive amount of energy may be input to the system as drug delivery commences. As another example, as higher viscosity drugs are delivered via autoinjectors, the requisite spring forces will likely increase. Springs with higher spring constants may transmit more force to the drug product and primary container. Various physical characteristics of a spring may affect the spring rate, and thus the spring force, such as wire diameter of the spring, mean diameter of the spring, the number of spring coils, and the spring material. However, as spring force increases the likelihood of damage to and/or failure of other components may increase. Therefore, it may be desirable and/or advantageous to include device components that permit flexibility in spring design and/or that facilitate the use of springs with different physical characteristics with the remaining device components.

The present disclosure sets forth drug delivery devices embodying advantageous alternatives to existing drug delivery devices, and that may address one or more of the challenges or needs mentioned herein.

### SUMMARY

One aspect of the present disclosure provides a drug delivery device with a housing defining a longitudinal axis and having an opening and a drug storage container including a barrel, a stopper and a delivery member, the stopper movably positioned within the barrel and the delivery member positioned at a distal end of the barrel and having an insertion end configured to extend at least partially through the opening during a delivery state. The device may also include plunger moveable toward the distal end of the drug storage container to engage the stopper and expel a drug from the drug storage container through the delivery member, the plunger including a body portion and a shock absorbing portion. The device may further include a plunger biasing member coupled with the plunger and configured to urge the plunger toward the distal end of the drug storage container.

The shock absorbing portion may include a collapsible portion, which may include at least one collapsible leg, at least two collapsible legs, or at least four collapsible legs.

The shock absorbing portion may include a foot portion coupled with the collapsible portion. The foot may define a distal end of the plunger configured to engage the stopper and wherein the collapsible portion is positioned between and operatively couples the foot and the plunger body portion.

The shock absorbing portion may have an extended position defining a first distance between the foot and the plunger body portion and a collapsed position defining a second distance between the foot and the plunger body, wherein the first distance is larger than the second distance.

The shock absorbing portion may include a hard stop defining the collapsed position. The hard stop may include a pair of protrusions extending towards each other along the longitudinal axis. The hard stop may include two pairs of protrusions, each extending towards each other along the longitudinal axis.

The body portion of the plunger may have a hollow tubular shape. The body portion may include a non-metal material, such as a thermoplastic material.

Another aspect of the present disclosure provides a plunger configured for use in a drug delivery device. The plunger may include a body portion having a proximal end and a distal end and a shock absorbing portion positioned adjacent to the distal end of the body portion, where the shock absorbing portion including at least one collapsible leg.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 is a perspective view of an exemplary drug delivery device in accordance with various embodiments;

FIG. 2 is a cross-sectional view of the drug delivery device in FIG. 1;

FIG. 3 is a cross-sectional view of the drug delivery device in FIG. 1, similar to the view in FIG. 2 but focused on a portion of the device showing a distal end of the plunger rod and the stopper;

FIG. 4 is a front view of a distal portion of another embodiment of a plunger rod in accordance with various embodiments, wherein the plunger rod is in an extended configuration;

FIG. 5 is a front view of a distal portion of the plunger rod shown in FIG. 3, wherein the plunger rod is in a collapsed configuration.

### DETAILED DESCRIPTION

The present disclosure generally relates to drug delivery devices operable by a user for administering a drug, or in the case where a patient is the user, self-administering a drug. Various features are disclosed for streamlining, simplifying, automating and/or facilitating certain aspects of drug delivery, such as those utilized in auto-injectors, on-body injectors, or other automatic or partially automatic drug delivery devices (collectively autoinjectors or auto-injectors). For example, these features may include automatically covering a needle in a pre-delivery and/or post-delivery state, automatically inserting a needle and/or a cannula into a user, automatically activating a drive mechanism, automatically indicating to the user that drug delivery is complete, among other features. Although known drug delivery devices incorporate a separate or independently operable mechanism to realize each of its automated features, the present disclosure includes eliminating and/or combining at least some of these features and/or providing device components that permit flexibility in device design. For example, the device may include components that permit flexibility in spring design and/or that facilitate the use of springs with different physical characteristics with the remaining device components. As another example, the device may include components that reduce the part number, part complexity, overall weight of the device, and/or overall complexity of the device. For example, the present disclosure may include a plunger moveable toward the distal end of the drug storage container to expel a drug from the drug storage container through the delivery member, where the plunger includes a body portion and a shock absorbing portion. The present disclosure may also include a plunger biasing member, where the plunger biasing member is configured to urge the plunger toward the distal end of the drug storage container.

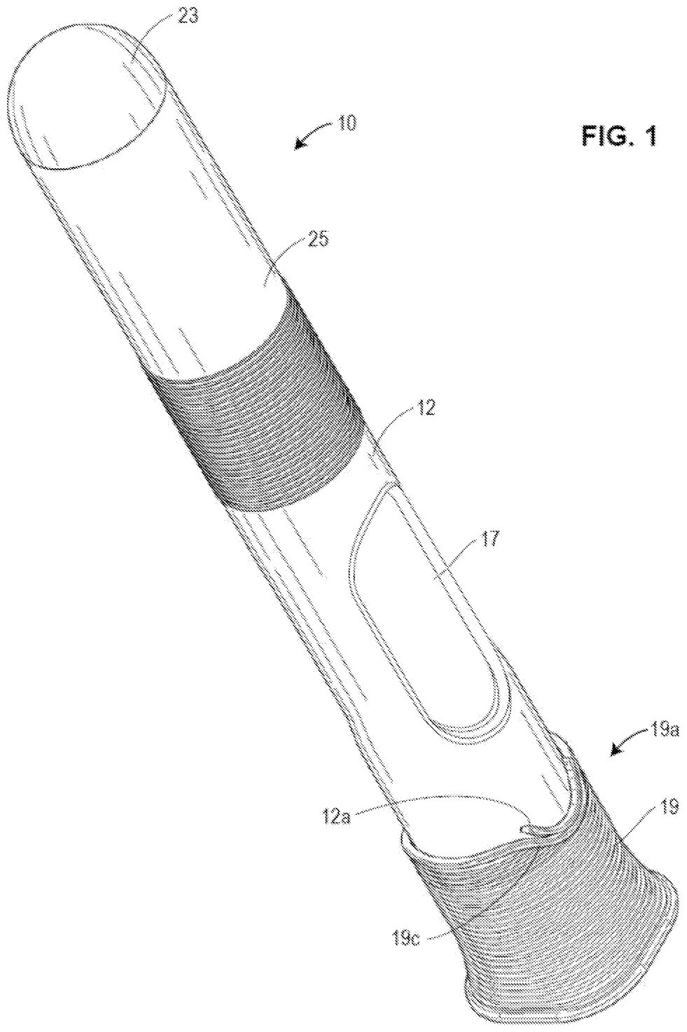

FIGS. 1-3 illustrate several views of an embodiment of a drug delivery device 10 for delivering a drug, which may also be referred to herein as a medicament or drug product. The drug may be, but is not limited to, various biologicals such as peptides, peptibodies, or antibodies. The drug may be in a fluid or liquid form, although the disclosure is not limited to a particular state.

Various implementations and configurations of the drug delivery device 10 are possible. The present embodiment of the drug delivery device 10 is configured as a single-use, disposable injector. In other embodiments, the drug delivery device 10 may be configured as multiple-use reusable injector. The drug delivery device 10 is operable for self-administration by a patient or for administration by caregiver or a formally trained healthcare provider (e.g., a doctor or nurse). The exemplary the drug delivery devices shown in the figures may take the form of an autoinjector or pen-type injector, and, as such, may be held in the hand of the user over the duration of drug delivery, but may also or alternatively be suitable for other drug delivery devices and/or configurations.

The configuration of various components included in the drug delivery device 10 may depend on the operational state of the drug delivery device 10. The drug delivery device 10 may have a pre-delivery or storage state, a delivery or dosing state, and a post-delivery state, although fewer or more states are also possible. For example, each state may have several sub-states or stages. The pre-delivery state may correspond to the configuration of the drug delivery device 10 subsequent to assembly and prior to activation by the user. In some embodiments, the pre-delivery state may exist in the time between when the drug delivery device 10 leaves a manufacturing facility and when a patient or user activates a drive mechanism 30 of the drug delivery device 10. This includes the moments in time after the user has removed the drug delivery device 10 from any secondary packaging and prior to positioning the drug delivery device 10 against the injection site. The delivery state may correspond to the configuration of the drug delivery device 10 while drug delivery, also referred to herein as dosing, is in progress. The post-delivery state may correspond to the configuration of the drug delivery device 10 after drug delivery is complete and/or when a stopper is arranged in an end-of-dose position in a drug storage container.

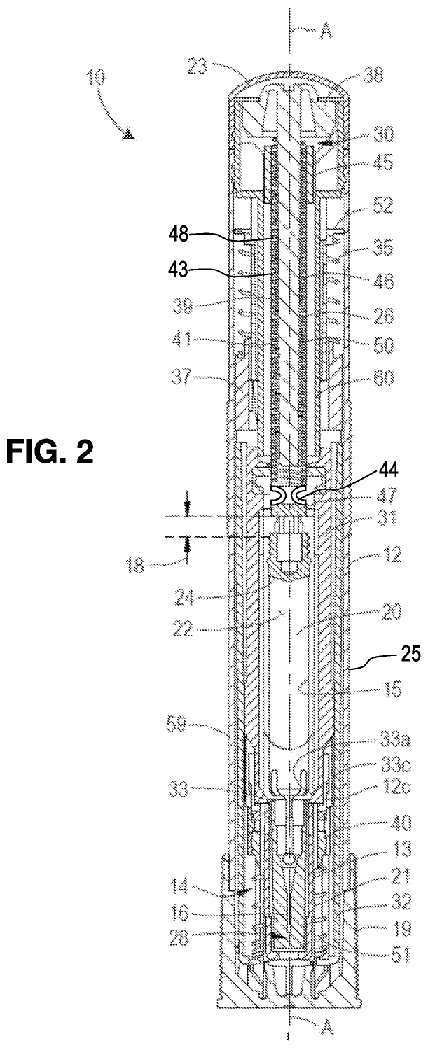

As shown in FIGS. 1 and 2, the drug delivery device 10 includes an outer casing or housing 12. In some embodiments, the housing 12 may be sized and dimensioned to enable a person to grasp the injector 10 in a single hand. The housing 12 may have a generally elongate shape, such as a cylindrical shape, and extend along a longitudinal axis A between a proximal end and a distal end. An opening 14 may be formed in the distal end to permit an insertion end 28 of a delivery member 16 to extend outside of the housing 12. A transparent or semi-transparent inspection window 17 may be positioned in a wall of the housing 12 to permit a user to view component(s) inside the drug delivery device 10, including a drug storage container 20. Viewing the drug storage container 20 through the window 17 may allow a user to confirm that drug delivery is in progress and/or complete. A removable cap 19 may cover the opening 14 prior to use of the drug delivery device 10, and, in some embodiments, may including a gripper 13 configured to assist with removing a sterile barrier 21 (e.g., a rigid needle shield (RNS), a non-rigid needle shield (nRNS), etc.) mounted on the insertion end 28 of the delivery member 16. The gripper 13 may include one or more inwardly protruding barbs or arms that frictionally or otherwise mechanically engage the sterile barrier 21 to pull the sterile barrier 21 with the removable cap 19 when the user separates the removable cap 19 from the housing 12. Thus, removing the removable cap 19 has the effect of removing the sterile barrier 21 from the delivery member 16. As shown in FIG. 1, the removable cap 19 and/or the housing 12 may include cap camming features such as cap camming feature 19c and housing camming feature 12a that are configured to convert rotational motion of the removable cap 19 into longitudinal motion of the same, thereby facilitating cap removal. As a more specific example, upon rotation of the removable cap 19, relative movement of the camming features 12a, 19c urge the removable cap 19 along the longitudinal axis A in the distal direction (downward in FIG. 2).

As shown in FIG. 2, the drive mechanism 30 may be disposed partially or entirely within the housing 12. Generally, the drive mechanism 30 may be configured to store energy and, upon or in response to activation of the drive mechanism 30 by the user, release or output that energy to drive the plunger 26 to expel the drug 22 from the drug storage container 20 through the delivery member 16 into the patient. In the present embodiment, the drive mechanism 30 is configured to store mechanical potential energy; however, alternative embodiments of the drive mechanism 30 may be configured differently, for example, with the drive mechanism 30 storing electrical or chemical potential energy. Generally, upon activation of the drive mechanism 30, the drive mechanism 30 may convert the potential energy into kinetic energy for moving the plunger 26. As best illustrated in FIG. 2, in one embodiment, the drive mechanism 30 includes the plunger biasing member 50, a hollow rod 46 for supporting the plunger biasing member 50, a plunger biasing member seat 38, the releaser member 52, a plunger guide 60, an extender biasing member 35, and a guard extension 37. The plunger biasing member 50 may include a compression spring (e.g., a helical compression spring) which is initially retained in an energized state. In the energized state, the plunger biasing member 50 may be compressed such that its axial length is shorter than it would be in a natural or de-energized state. When released, the plunger biasing member 50 may try to expand to its natural axial length, and as a consequence, exert a biasing force pushing the plunger 26 in the distal direction. The plunger 26 may also include a top ring 45 defining a proximal end of the plunger 26. The top ring 45 may include one or more flanges or projections which extend radially outwardly from a central portion of the top ring 45 to facilitate a locked configuration and a released configuration for the plunger.

As best shown in FIGS. 1-2, in one embodiment the device 10 include a housing 12 may include two separate and interconnected structures: a rear end cap 23 (e.g., a rear cover) at the proximal end of the drug delivery device 10; and a tubular housing 25 extending substantially completely along the length of the drug delivery device 10 and defining the opening 14. The housing 12 shown in FIG. 2 also includes a guard member 32 that surrounds the delivery member 16 and protects against or reduces the likelihood of unintended or premature needle stick. Additionally or alternatively, the housing 12 may include fewer or more components, such as a two-piece tubular housing having front and rear portions. The tubular housing 25 may have a hollow and generally cylindrical or tubular shape, and the rear end cap 23 may have a generally hemispherical shape or a hollow cylindrical shape with an open end and a closed off end. In some embodiments, the rear end cap 23 and the tubular housing 25, and any components to be positioned therein, may be assembled together to define different sub-assemblies, such as the drive mechanism 30. In some embodiments, the different sub-assemblies are assembled independently of each other and then later combined with one another, as well as with the drug storage container 20, to form the fully-assembled drug delivery device 10. In certain such embodiments, some or all of the foregoing phases of assembly may occur in different manufacturing facilities or environments. In alternative embodiments, the housing 12 may be constructed in one piece, such that the housing 12 is defined by a single, monolithic structure that integrates a rear cap and tubular housing in a single component.

The drug storage container 20 is disposed within an interior space of the housing 12 and is configured to contain a drug 22. The drug storage container 20 may be pre-filled and shipped, e.g., by a manufacturer, to a location where the drug storage container 20 is combined with a remainder of the drug delivery device 10. For example, the drug 22 may be distributed and/or provided to patients in more than one use case, such as a as a pre-filled syringe or as an autoinjector including a pre-filled syringe. By utilizing the same or similar syringe components in either case, at least some of above steps such as filling, labeling, packaging, shipping, and distribution may be streamlined or simplified for two different use cases. As a another example, in the event that multiple use cases utilize some or all of the same syringe components, some regulatory pathways to marketing and/or distributing the drug may be streamlined and/or simplified for at least one of the multiple use cases.

The housing 12 may be pre-loaded with the drug storage container 20, e.g., by a manufacturer, or alternatively, loaded with the drug storage container 20 by a user prior to use of the drug delivery device 10. The drug storage container 20 may include a rigid wall defining an internal bore or reservoir. The wall may be made of glass or plastic. A stopper 24 may be moveably disposed in the drug storage container 20 such that it can move in a distal direction along the longitudinal axis A between proximal end and a distal end of the drug storage container 20. The stopper 24 may be constructed of rubber or any other suitable material. The stopper 24 may slidably and sealingly contact an interior surface 15 of the wall of the drug storage container 20 such that the drug 22 is prevented or inhibited from leaking past the stopper 24 when the stopper 24 is in motion. Distal movement of the stopper 24 expels the drug 22 from the reservoir of the drug storage container 20 into the delivery member 16. The proximal end of the drug storage container 20 may be open to allow the plunger 26 to extend into the drug storage container 20 and push the stopper 24 in the distal direction. In the present embodiment, the plunger 26 and the stopper 24 are initially spaced from each other by a gap 18 (FIG. 2). Upon activation of a drive mechanism 30, the plunger 26 moves in the distal direction to close the gap and comes into contact with the stopper 24. Subsequent distal movement of the plunger 26 drives the stopper 24 in the distal direction to expel the drug 22 from the drug storage container 20. In alternative embodiments, the stopper 24 and the plunger 26 may initially be in contact with one another or coupled to one another, e.g., via a threaded coupling, such that they move together jointly from the start of movement of the plunger 26. Once the stopper 24 is in motion, it may continue to move in the distal direction until it contacts a proximally-facing portion of the interior surface 15 of the wall of the drug storage container 20. This position of the stopper 24 may be referred to as the end-of-dose or end-of-delivery position, and may correspond to when delivery of the drug 22 to the patient is complete or substantially complete.

The plunger 26 may generally include a body portion 39 and a shock absorbing portion 44 configured to dampen or absorb impact force when the plunger 26 impacts the stopper 24 during activation of the device 10. The plunger 26 may also include a foot 47 defining a distal end of the plunger and configured to engage the stopper 24 during activation of the device 10.

The body portion 39 of the plunger 26 may have a hollow and generally cylindrical or tubular shape such as a hollow rod 46. As a more specific example, the hollow rod 46 may have inner surface 43 may define an interior space sized to receive a plunger biasing member 50 therein. It is generally desirable to minimize a thickness of the hollow rod 46, to the extent possible and without compromising the integrity of the plunger 26, so as to maximize an inner diameter of the plunger 26. This allows a larger diameter plunger biasing member 50 to fit within the interior space of the plunger 26, which, in turn, allows for a more powerful plunger biasing member 50. As a more specific example, the thickness of the hollow rod 46 may be less than 2 mm. As another more specific example, the thickness of the hollow rod may be less than 1 mm. As another more specific example, the thickness of the hollow rod may be less than 0.6 mm. As another more specific example, the thickness of the hollow rod may be less than 0.3 mm. As another more specific example, the thickness of the hollow rod may be less than 0.2 mm. As another more specific example, the thickness of the hollow rod may be less than 0.1 mm. As another more specific example, the thickness of the hollow rod may be less than 0.05 mm.

The hollow rod 46 may additionally or alternatively facilitate and/or provide more flexibility in spring design. For example, it may be desirable or advantageous to use the device with different springs depending on the characteristics of the drug and/or the desired drug delivery profile. For example, a higher viscosity drug may require a spring with a higher spring rate and/or spring force and it thus may be desirable or advantageous to have flexibility in physical characteristics of the spring. As a more specific example, various physical characteristics of a spring may affect the spring rate, and thus the spring force, such as wire diameter of the spring (typically increasing the wire diameter increases the spring rate), mean diameter of the spring (typically increasing the mean diameter decreases the spring rate), the number of spring coils (typically increasing the number of coils increases the spring rate), and the spring material. These physical characteristics may be adjusted to deliver different spring rates, while also potentially adjusting the thickness of the hollow rod 46, to maintain a constant or relatively constant outer diameter of the overall plunger 26 so as to keep constant the remaining parts of the device, such as the plunger guide 60 and the stopper 24. The hollow rod 46 may additionally or alternatively facilitate and/or provide more longitudinal stability for the plunger biasing member 50, such as by preventing or reducing buckling or other transverse movement.

The body portion 39 shown in the figures includes a metal hollow rod 46 made of metal and an overmolding 48 made of thermoplastic. Alternatively, the body portion 39 may be made of any suitable material, such as metal and/or plastic. It may be advantageous for the hollow rod 46 to be made of metal, such as steel or aluminum, for the purposes of minimizing the thickness of the hollow rod 46. For example, a metal hollow rod 46 may have sufficient axial strength and/or buckle resistance for use in the device if the annular wall thickness is greater than 0.05 mm. Conversely, a plastic annular wall 39 may have sufficient axial strength and/or buckle resistance for use in the device if the annular wall 39 thickness is greater than 1 mm. The metal hollow rod 46 and thermoplastic overmolding 48 may permit the plunger 26 to have a sufficient axial strength while also giving the plunger 26 a desirable surface finish that minimizes friction between the plunger 26 and the drug storage container 20. Additionally or alternatively, the thermoplastic overmolding material may facilitate manufacture and/or performance of other features of the plunger rod 26, such as the shock absorbing portion 44 and/or the foot 47.

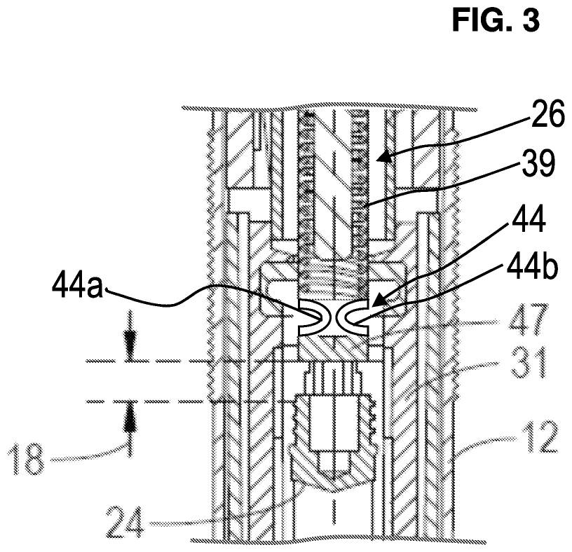

As shown in FIGS. 2-3, the shock absorbing portion 44 includes a collapsible portion that dampens and/or absorbs force from impact between the plunger rod 26 and the stopper 24. As a more specific example, the collapsible portion shown in FIGS. 2-3 includes a pair of collapsible legs 44a, 44b that each have a general “U” shape when in a first (extended) position and “flattened U” shape when in a second (collapsed) position. The collapsible legs 44a, 44b may elastically deform to absorb force. Additionally or alternatively, the collapsible legs 44a, 44b may plastically deform to absorb force. Additionally or alternatively, the collapsible legs 44a, 44b may deform in a manner that is part elastic deformation and part plastic deformation. The collapsible portion may have any suitable number of legs, such as one, two, three, four, or more. The collapsible legs may have any suitable shape, such as a “V” shape, a “W” shape, a coil spring shape, a leaf spring shape, or any suitable shape.

The shock absorbing portion 44 may be made of any suitable material, such as thermoplastic, other types of plastic, rubber, metal, or any suitable material. As discussed above, the shock absorbing portion 44 may be an integral part of an overmold that surrounds the metal hollow rod and may be formed via a thermoplastic molding process. The thickness, shape, and material of the components of the shock absorbing portion 44 will affect the stiffness thereof, thereby permitting variance in the amount of force absorbed by the shock absorbing portion, as is discussed below in more detail.

Additionally or alternatively, the shock absorbing portion 44 may be formed of an elastomeric material that includes inherent damping properties. In such an instance, the shock absorbing portion 44 may have a diameter and outer shape similar to the body portion 39 of the plunger 26, such as an elastomeric disc or cylinder at the distal end of the plunger 26.

As discussed above, the plunger 26 may include a foot 47 that defines a distal end of the plunger 26. The foot 47 may be configured to engage the stopper 24 such that the collapsible portion 44 is positioned between and operatively couples the foot 47 and the plunger body portion 39 of the plunger 26. The foot may also be an integral part of an overmold that surrounds the metal hollow rod and may be formed via a thermoplastic molding process. The foot may be generally cylindrical, not unlike a disc-shape, such as to suitably engage the stopper 24 upon activation of the device 10.

As a more specific example, during activation of the device 10, as the plunger 26 travels distally, the gap 18 between the foot 47 of the plunger 26 and the stopper 24 rapidly closes and the foot 47 contacts the stopper 24. The device 10 is designed such that plunger 26 is traveling with a force sufficient to drive the stopper 24 in the distal direction and urge the drug 22 from the delivery member 16. At the same time, the device 10 is also designed such as to reduce or eliminate the likelihood of glass breakage, undesirable forces acting on the patient, and/or undesirable impact vibration or sound due to interaction between the base 47 and the stopper 24. For example, the plunger biasing member 50 design parameters may be designed to meet these two sets of design goals. As another example, the shock absorbing portion 44 may dampen the forces between the foot 47 and the stopper 24. As a consequence, the velocity of the plunger 26 may be reduced during the initial contact between the plunger 26 and the stopper 24, thereby reducing the impact force between the respective components and reducing the likelihood of structural damage to the drug storage container 20 and/or providing a more comfortable injection for the user.

In some embodiments, a volume of the drug 22 included in the reservoir of the drug storage container 20 may be equal to 1 mL, or equal to approximately (e.g., +10%) 1 mL, or equal to 2.5 mL, or equal to approximately (e.g., +10%) 2.5 mL, or equal to 3 mL, or equal to approximately (e.g., +10%) 3 mL, or less than or equal to approximately (e.g., +10%) 1 mL, or less than or equal to approximately (e.g., +10%) 2 mL, or less than or equal to approximately (e.g., +10%) 3 mL, or less than or equal to approximately (e.g., +10%) 4 mL, or less than approximately (e.g., +10%) 5 mL, or less than or equal to approximately (e.g., +10%) 10 mL, or within a range between approximately (e.g., +10%) 1-10 mL, or within a range between approximately (e.g., +10%) 1-5 mL, or within a range between approximately (e.g., +10%) 1-4 mL, or within a range between approximately (e.g., +10%) 1-3 mL, or within a range between approximately (e.g., +10%) 1-2.5 mL.

The delivery member 16 is connected or operable to be connected in fluid communication with the reservoir of the drug storage container 20. A distal end of the delivery member 16 may define the insertion end 28 of the delivery member 16. The insertion end 28 may include a sharpened tip of other pointed geometry allowing the insertion end 28 to pierce the patient's skin 5 and subcutaneous tissue during insertion of the delivery member 16. The delivery member 16 may be hollow and have an interior passageway. One or more openings may be formed in the insertion end 28 to allow drug to flow out of the delivery member 16 into the patient.

In one embodiment, the drug storage container 20 may be a pre-filled syringe and has a staked, hollow metal needle for the delivery member 16. Here, the needle is fixed relative to the wall of the drug storage container 20 and may be in permanent fluid communication with the reservoir of the drug storage container 20. In other embodiments, the needle may be coupled to the drug storage container 20 via a Luer Lock or other suitable connection. In yet other embodiments, the drug storage container 20 may be a needle-less cartridge, and, as such, initially may not be in fluid communication with the delivery member 16. In such embodiments, the drug storage container 20 may move toward a proximal end of the delivery member 16, or vice versa, during operation of the drug delivery device 10 such that the proximal end of the delivery member 16 penetrates through a septum covering an opening in the drug storage container 20 thereby establishing fluid communication between the reservoir of the drug storage container 20 and the delivery member 16.

The container holder 31 may have a hollow and generally cylindrical or tubular shape centered about the longitudinal axis A, and the drug storage container 20 may be disposed partially or entirely within the container holder 31. A distal end of the container holder 31 may include an inwardly protruding flange 33 abutting against a shoulder portion 20a of the drug storage container 20, thereby preventing distal movement of the drug storage container 20 during actuation of the plunger 26. The container holder 31 may include flanges 33, each of which includes an arcuate, sloped surface 33a that substantially matches the arcuate shape of a shoulder portion of the drug storage container. The housing 12 may include a plurality of lock slots 12c that each receive respective lock ridges 33c of the container holder 31 to prevent and/or restrict relative movement between the respective components 12, 31.

After drug delivery is complete and the guard member 32 has been re-deployed to the extended position, it may be desirable to lock the guard member 32 in the extended position to prevent subsequent user contact with the insertion end 28 of the delivery member 16 and/or to prevent re-use of the drug delivery device 10. Pursuant to these ends, some embodiments of the drug delivery device 10 may include a lock ring 40 configured to selectively rotate, depending on the axial position of the guard member 32, in order to lock the guard member 32 in the extended position once the guard member 32 has moved from the retracted position to the extended position. The device may also include a lock ring biasing member 51 that urges the guard member 32 in the outward (distal) direction and facilitates movement of the guard member 32 to the extended position, thereby covering the insertion end 28 of the delivery member 16.

More information on these features, and other features disclosed in the present disclosure, are shown and described in more detail in U.S. application Ser. No. 17/035,851, filed on Sep. 29, 2020 and claiming priority to U.S. application No. 62/960,996 and U.S. application No. 62/960,996, each of which is hereby incorporated by reference.

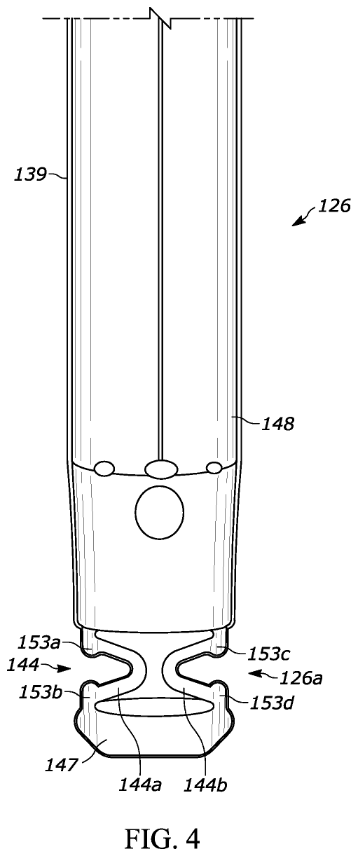

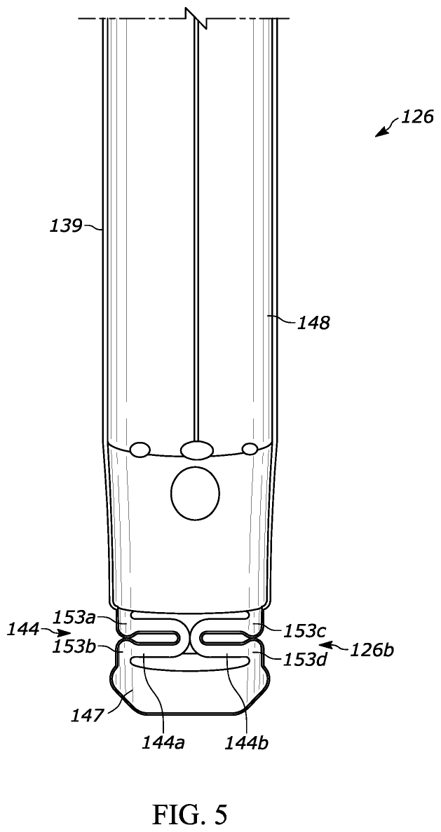

FIGS. 4 and 5 show another plunger rod 126 in accordance with various embodiments. In FIG. 4, the plunger rod 126 is shown in an extended configuration 126a and in FIG. 5 the plunger rod 126 is shown in a collapsed configuration 126b. The plunger rod shown in FIGS. 4-5 includes a body portion 139 with a metal hollow rod made of metal and an overmolding 148 made of thermoplastic. The plunger rod also includes a shock absorbing portion 144 that dampens and/or absorbs force from impact between the plunger rod 126 and the stopper. As a more specific example, the collapsible portion shown in FIGS. 4-5 includes a pair of collapsible legs 144a, 144b that each have a general “U” shape when in a first (extended) position 126a and “flattened U” shape when in a second (collapsed) position 126b. The collapsible legs 144a, 144b may elastically deform to absorb force. Additionally or alternatively, the collapsible legs 144a, 144b may plastically deform to absorb force. The collapsible legs may have any suitable shape, such as a “V” shape, a “W” shape, a coil spring shape, a leaf spring shape, or any suitable shape.

The shock absorbing portion 144 shown in FIGS. 4-5 includes at least one hard stop 153 that defines the collapsed position 126b. As a more specific example, the shock absorbing portion 144 includes two pairs of opposing protrusions 153a, 153b, 153c, 153d, each extending towards each other along the longitudinal axis. When the plunger rod 126 is in the collapsed configuration 126b, the hard stops 153a, 153b, 153c, 153d contact each other and prohibit further compression, thereby defining the collapsed portion.

The shock absorbing portion may reduce the impact energy of the plunger/stopper interaction by approximately 1%, approximately 2%, approximately 3%, approximately 4%, approximately 5%, approximately 6%, approximately 8%, approximately 10%, approximately 12%, approximately 15%, or any suitable amount. As a more specific example, a 20N spring with a free height of 3 mm (i.e., if the gap 18 shown in FIG. 2 is 3 mm) then the plunger will impact the stopper with 0.06 Joules (3 mm*20 N=0.06 Joules) without a shock absorbing portion. However, the shock absorbing portion may reduce the impact energy by about 10% (e.g., by about 0.006 Joules). As another example, a 20N spring with a free height of 15 mm (i.e., if the gap 18 shown in FIG. 2 is 15 mm) then the plunger will impact the stopper with 0.3 Joules (15 mm*20 N=0.3 Joules) without a shock absorbing portion. However, the shock absorbing portion may reduce the impact energy by about 2% (e.g., by about 0.006 Joules). The shock absorbing portion may be constructed with different parameters to give it more or less energy reducing capability, as is desired, such as by making the collapsible legs stiffer or less stiff.

From the foregoing, it can be seen that the present disclosure advantageously provides a streamlined design for a drug delivery device having automated features. Various mechanisms and components of the drug delivery device may interact with each other in synergistic ways so as to limit the number of moving parts required by the drug delivery device, thereby improving the reliability of the drug delivery device and saving costs, as well as providing other benefits and advantages.

As will be recognized, the devices and methods according to the present disclosure may have one or more advantages relative to conventional technology, any one or more of which may be present in a particular embodiment in accordance with the features of the present disclosure included in that embodiment. Other advantages not specifically listed herein may also be recognized as well.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIIa receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL 15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL 12 mAb (ABT-874); anti-IL 12/IL23 mAb (CNTO 1275); anti-IL 13 mAb (CAT-354); anti-IL2Rα mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BITE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRVIII) BITE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BITE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing defining a longitudinal axis and having an opening;
a drug storage container including a barrel, a stopper and a delivery member, the stopper movably positioned within the barrel, the delivery member positioned at a distal end of the barrel and having an insertion end configured to extend at least partially through the opening during a delivery state;
a plunger moveable toward the distal end of the drug storage container to engage the stopper and expel a drug from the drug storage container through the delivery member, the plunger including a body portion and a shock absorbing portion; and
a plunger biasing member coupled with the plunger and configured to urge the plunger toward the distal end of the drug storage container.

2. The drug delivery device of claim 1, wherein the shock absorbing portion includes a collapsible portion.

3. The drug delivery device of claim 2, wherein the collapsible portion includes at least one collapsible leg, at least two collapsible legs, or at least four collapsible legs.

4. (canceled)

5. (canceled)

6. The drug delivery device of claim 2, wherein the shock absorbing portion includes a foot coupled with the collapsible portion.

7. The drug delivery device of claim 6, wherein the foot defines a distal end of the plunger configured to engage the stopper and wherein the collapsible portion is positioned between and operatively couples the foot and the plunger body portion.

8. The drug delivery device of claim 7, wherein the shock absorbing portion has an extended position defining a first distance between the foot and the plunger body portion and a collapsed position defining a second distance between the foot and the plunger body, wherein the first distance is larger than the second distance.

9. The drug delivery device of claim 8, wherein the shock absorbing portion includes a hard stop defining the collapsed position.

10. The drug delivery device of claim 9, wherein the hard stop includes at least one pair of protrusions extending towards each other along the longitudinal axis.

11. (canceled)

12. The drug delivery device of claim 1, wherein the body portion has a hollow tubular shape.

13. The drug delivery device of claim 1, wherein the body portion includes a non-metal material, and wherein the shock absorbing portion is defined by the non-metal material.

14. (canceled)

15. The drug delivery device of claim 13, wherein the non-metal material is a thermoplastic material.

16. A plunger configured for use in a drug delivery device, the plunger comprising:
a body portion having a proximal end and a distal end;
a shock absorbing portion positioned adjacent to the distal end of the body portion, the shock absorbing portion including at least one collapsible leg.

17. (canceled)

18. (canceled)

19. The plunger of claim 16, wherein the shock absorbing portion includes a foot coupled with the at least one collapsible leg.

20. The plunger of claim 19, wherein the foot is disposed at the distal end of the body portion and wherein the at least one collapsible leg is positioned between and operatively couples the foot and a remainder of the body portion.

21. The plunger of claim 20, wherein the shock absorbing portion has an extended position defining a first distance between the foot and the remainder of the body portion and a collapsed position defining a second distance between the foot and the remainder of the body portion, wherein the first distance is larger than the second distance.

22. The plunger of claim 21, wherein the shock absorbing portion includes a hard stop defining the collapsed position.

23. The plunger of claim 22, wherein the hard stop includes at least one pair of protrusions extending towards each other along the longitudinal axis.

24. (canceled)

25. The plunger of claim 16, wherein the body portion has a hollow tubular shape.

26. The plunger of claim 16, wherein the body portion includes a non-metal material, and wherein the shock absorbing portion is defined by the non-metal material.

27. (canceled)

28. The plunger of claim 26, wherein the non-metal material is a thermoplastic material.
